(19) United States
(12) Patent Application Publication
(10) Pub. No.: US 2026/0198884 A1
(43) Pub. Date: Jul. 16, 2026

(54) SYSTEM AND METHOD FOR THRESHOLDING FOR X-RAY TECHNIQUE OPTIMIZATION

(71) Applicant: GE Precision Healthcare LLC, Waukesha, WI (US)

(72) Inventors: Chelsey Amanda Lewis, Brookfield, WI (US); Dean Michael Zsenak, Brookfield, WI (US); Franco John Rupcich, Wauwatosa, WI (US); John Howard Londt, Oconomowoc, WI (US); Robert Bujila, Delafield, WI (US)

(21) Appl. No.: 19/022,265

(22) Filed: Jan. 15, 2025

(51) Int. Cl. A61B 6/00; G06T 5/60

(52) U.S. Cl. CPC A61B 6/544; A61B 6/488; A61B 6/545; G06T 5/60

(57) ABSTRACT

A method includes receiving a prescription for a diagnostic X-ray scan of a subject with a medical imaging scanner, wherein diagnostic X-ray scan has a prescribed value for a target metric. The method includes determining a predicted value for the target metric for an altered prescription for the diagnostic X-ray scan based on alteration of the prescription by an automatic control exposure algorithm. The method includes receiving an input of a threshold value in a graphical user interface, wherein the threshold value is an acceptable percent deviation between the predicted value and the prescribed value for the target metric. The method includes determining whether the altered prescription for the diagnostic X-ray scan is sub-optimal based on a comparison of the threshold value to a percent deviation between the predicted value and the prescribed value for the target metric.

### BACKGROUND

The subject matter disclosed herein relates to imaging systems and, more particularly, to a system and a method for thresholding for X-ray technique optimization.

Volumetric medical imaging technologies use a variety of techniques to gather three-dimensional information about the body. For example, a computed tomography (CT) imaging system measures the attenuation of X-ray beams passed through a patient from numerous angles. Based upon these measurements, a computer is able to reconstruct cross-sectional images of the portions of a patient's body responsible for the radiation attenuation. As will be appreciated by those skilled in the art, these images are based upon separate examination of a series of angularly-displaced measurements. It should be pointed out that a CT system produces data that represents the distribution of linear attenuation coefficients of the scanned object. The data are then reconstructed to produce an image that is typically displayed on a screen and may be printed or reproduced on film.

In the context of CT scanning with automatic exposure control (AEC) prescriptions, a key element of the workflow is making adjustments to the current prescription to better optimize the technique toward the target metric (e.g., achieving the target noise index, target does, target tube current (e.g., target milliamperes (mA) or mAs), and so forth). It is common that users review the outputs of the AEC algorithms and determine whether the prescription must be adjusted (e.g., by reducing the pitch or rotation time to increase the dose or reduce the noise of the prescription). Additionally, in instances where the optimization of the prescription provides only a marginal benefit to diagnostic quality of the images it may be preferable to skip optimization.

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In one embodiment, a method for thresholding to optimize an X-ray technique is provided. The method includes receiving, via a processing system including one or more processors, a prescription for a diagnostic X-ray scan of a subject with a medical imaging scanner, wherein diagnostic X-ray scan has a prescribed value for a target metric. The method also includes determining, via the processing system, a predicted value for the target metric for an altered prescription for the diagnostic X-ray scan based on alteration of the prescription by an automatic control exposure algorithm. The method further includes receiving, via the processing system, an input of a threshold value in a graphical user interface, wherein the threshold value is an acceptable percent deviation between the predicted value and the prescribed value for the target metric. The method further includes determining, via the processing system, whether the altered prescription for the diagnostic X-ray scan is sub-optimal based on a comparison of the threshold value to a percent deviation between the predicted value and the prescribed value for the target metric.

In another embodiment, a system for thresholding to optimize an X-ray technique is provided. The system includes a medical imaging system. The medical imaging system includes a memory encoding processor-executable routines. The medical imaging system also includes a processing system including one or more processors and configured to access the memory and to execute the processor-executable routines, wherein the processor-executable routines, when executed by the processing system, cause the processing system to perform actions. The actions include receiving a prescription for a diagnostic X-ray scan of a subject with a medical imaging scanner, wherein diagnostic X-ray scan has a prescribed value for a target metric. The actions also include determining a predicted value for the target metric for an altered prescription for the diagnostic X-ray scan based on alteration of the prescription by an automatic control exposure algorithm. The actions further include receiving an input of a threshold value in a graphical user interface, wherein the threshold value is an acceptable percent deviation between the predicted value and the prescribed value for the target metric. The actions further include determining whether the altered prescription for the diagnostic X-ray scan is sub-optimal based on a comparison of the threshold value to a percent deviation between the predicted value and the prescribed value for the target metric.

In a further embodiment, a non-transitory computer-readable medium, the computer-readable medium including processor-executable code that when executed by a processing system including one or more processors, causes the processing system to perform actions. The actions include receiving a prescription for a diagnostic X-ray scan of a subject with a medical imaging scanner, wherein diagnostic X-ray scan has a prescribed value for a target metric. The actions also include determining a predicted value for the target metric for an altered prescription for the diagnostic X-ray scan based on alteration of the prescription by an automatic control exposure algorithm. The actions further include receiving an input of a threshold value in a graphical user interface, wherein the threshold value is an acceptable percent deviation between the predicted value and the prescribed value for the target metric. The actions further include determining whether the altered prescription for the diagnostic X-ray scan is sub-optimal based on a comparison of the threshold value to a percent deviation between the predicted value and the prescribed value for the target metric.

### BRIEF DESCRIPTION OF THE DRAWINGS

These and other features, aspects, and advantages of the disclosed subject matter will become better understood when the following detailed description is read with reference to the accompanying drawings in which like characters represent like parts throughout the drawings, wherein:

FIG. 1 is a combined pictorial view and block diagram of a computed tomography (CT) imaging system as discussed herein;

FIG. 2 is a schematic diagram of a computing device for performing the disclosed techniques, in accordance with aspects of the present disclosure;

FIG. 3 is a flow chart of a method for thresholding to optimize an X-ray technique, in accordance with aspects of the present disclosure;

FIG. 4 is a flow chart of a method for thresholding to optimize an X-ray technique (e.g., using user input for threshold value), in accordance with aspects of the present disclosure;

FIG. 5 is a flow chart of a method for thresholding to optimize an X-ray technique (e.g., using preset threshold value), in accordance with aspects of the present disclosure;

FIG. 6 is a portion of a graphical user interface illustrating a field for a threshold value, in accordance with aspects of the present disclosure;

FIG. 7 is a portion of a graphical user interface illustrating a field for a threshold value, in accordance with aspects of the present disclosure;

FIG. 8 is a schematic diagram illustrating different sources for setting a threshold value, in accordance with aspects of the present disclosure;

FIG. 9 is a schematic diagram of the effect of not having thresholding;

FIG. 10 is a schematic diagram of the effect of thresholding, in accordance with aspects of the present disclosure;

FIG. 11 is a schematic diagram of the effect of thresholding with automatic optimization, in accordance with aspects of the present disclosure;

FIG. 12 depicts an example graphical user interface utilizing thresholding and having a recommendation provided, in accordance with aspects of the present disclosure;

FIG. 13 depicts an example graphical user interface utilizing threshold and its features, in accordance with aspects of the present disclosure;

FIG. 14 is a schematic diagram of different levels of configuration for a graphical user interface that utilizes thresholding, in accordance with aspects of the present disclosure; and

FIG. 15 depicts an example of a graphical user interface having the field for the threshold value in FIG. 6, in accordance with aspects of the present disclosure.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers'specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C #, Pascal, Fortran, Perl, MATLAB, SAS, SPSS, JavaScript, AJAX, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

The present disclosure provides embodiments for a system and a method for thresholding to optimize an X-ray technique. The disclosed embodiments provide a system to accept user input (e.g., of a threshold value from a site or lead technologist) which is used by the system to identify whether optimization of the prescription for a scan (e.g., diagnostic X-ray scan) with a medical imaging scanner (e.g., CT scanner) is recommended or may be skipped (e.g., based on site tolerance) rather than relying on the operator to make that determination themselves. In particular, the disclosed embodiments provide a user interface that enables a user (e.g., site or lead technologist) to specify threshold value (e.g., percent deviation from a target value). If a prescription's quality metric (after alteration by one or more AEC algorithms of an AEC module) quality metric or target metric deviates from a target value by more than the threshold, the user is prompted to optimize (e.g., change one or more parameters) the prescription (e.g., as altered by the AEC algorithms).

The disclosed embodiments provide the user control over how much of a deviation from a target value of a quality metric or target metric may be accepted by the prescription. The disclosed embodiments provide a streamlined workflow. The disclosed embodiments reduce the time to interpret the output of AEC algorithms. The disclosed embodiments provide a higher consistency in the interpretation of the output of the AEC algorithms among different operators. The disclosed embodiments provide a higher consistency in an individual operator's interpretation of the output of AEC algorithms.

In some embodiments, the systems and methods include receiving a prescription (e.g., parameters for conducting a specific scan) for a diagnostic X-ray scan of a subject with a medical imaging scanner, wherein diagnostic X-ray scan has a prescribed value for a target metric. In some embodiments, the systems and methods also include determining a predicted value for the target metric for an altered prescription for the diagnostic X-ray scan based on alteration of the prescription by an automatic control exposure algorithm. In some embodiments, the systems and methods further include receiving an input of a threshold value in a graphical user interface, wherein the threshold value is an acceptable percent deviation between the predicted value and the prescribed value for the target metric. In some embodiments, the systems and methods include determining whether the altered prescription for the diagnostic X-ray scan is sub-optimal based on a comparison of the threshold value to a percent deviation between the predicted value and the prescribed value for the target metric.

In certain embodiments, the systems and methods include providing a user-perceptible indication on the graphical user interface that the percent deviation exceeds the threshold value for the target metric when the percent deviation exceeds the threshold value for the target metric. In certain embodiments, the user-perceptible indication includes a recommendation to change one or more parameters of the altered prescription to optimize the altered prescription. In certain embodiments, the user-perceptible indication includes an option to accept the altered prescription even though the altered prescription for the diagnostic X-ray scan is sub-optimal, and the systems and methods further include receiving user input via the graphical user interface to accept the option. In certain embodiments, the systems and methods include automatically accepting, via the processing system, the altered prescription when the percent deviation is equal to or less than the threshold value for the target metric. In certain embodiments, the systems and methods include providing a user-perceptible prompt on the graphical user interface to accept the altered prescription when the percent deviation is equal to or less than the threshold value for the target metric and receiving user input via the user-perceptible prompt on the graphical user interface to accept the altered prescription.

In certain embodiments, receiving the input of the threshold value in the graphical user interface includes receiving user input of the threshold value. In certain embodiments, the systems and methods include altering the threshold value inputted via user input to a system threshold value when the threshold value inputted via the user input exceeds the system threshold value, wherein the system threshold value is a value that results in an accepted tolerance for the target metric across all protocols, all series, and all groups for scans with the medical imaging scanner.

In certain embodiments, receiving the input of the threshold value in the graphical user interface includes automatically obtaining a preset threshold value. In certain embodiments, the preset threshold value is a site level threshold value for a site including one or more medical imaging scanners including the medical imaging scanner, a system level threshold value for the medical imaging scanner, a protocol level threshold value specific to a particular medical imaging protocol with the medical imaging scanner, a series level threshold value specific to scanning one or more groups with the particular medical imaging protocol with the medical imaging scanner, or a group level threshold value specific to scanning an individual with the particular medical imaging protocol with the medical imaging scanner. In certain embodiments, the graphical user interface is configurable by a user to present the graphical user interface in a first manner when the percent deviation exceeds the threshold value for the target metric and to present the graphical user interface in a second manner different from the first manner when the percent deviation is equal to or less than the threshold value for the target metric. In certain embodiments, the target metric includes a noise index. In certain embodiments, the target metric includes a ratio between an average tube current of the prescription and an average tube current determined to be optimal by the automatic control exposure algorithm.

Although the following discusses the disclosed embodiments with regard to CT imaging systems, the techniques described herein may apply to other types of medical imaging systems. For example, the disclosed techniques may apply to an MRI system or a nuclear medicine imaging system such as a PET imaging system or a SPECT imaging system. The disclosed techniques may also apply to medical imaging systems having a combination of the above medical imaging modalities.

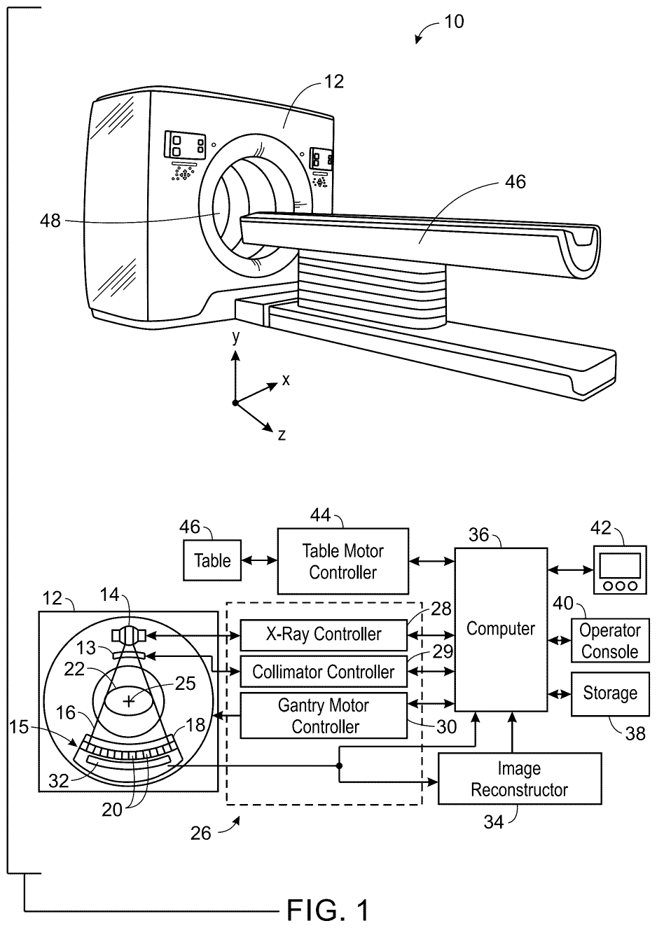

With the preceding in mind and referring to FIG. 1, a computed tomography (CT) imaging system 10 is shown, by way of example. The CT imaging system 10 includes a gantry 12. The gantry 12 has an X-ray source 14 that projects a beam of X-rays 16 toward a detector assembly 15 on the opposite side of the gantry 12. The X-ray source 14 projects the beam of X-rays 16 through a pre-patient collimator assembly 13 that determines the size and shape of the beam of X-rays 16. The detector assembly 15 includes a collimator assembly 18 (a post-patient collimator assembly), a plurality of detector modules 20 (e.g., detector elements or sensors), and data acquisition systems (DAS) 32. The plurality of detector modules 20 detect the projected X-rays that pass through a subject or object 22 being imaged, and DAS 32 converts the data into digital signals for subsequent processing. Each detector module 20 in a conventional system produces an analog electrical signal that represents the intensity of an incident X-ray beam and hence the attenuated beam as it passes through the subject or object 22. During a scan to acquire X-ray projection data, gantry 12 and the components mounted thereon rotate about a center of rotation 25 (e.g., isocenter) so as to collect attenuation data from a plurality of view angles relative to the imaged volume.

Rotation of gantry 12 and the operation of X-ray source 14 are governed by a control system 26 of CT imaging system 10. Control system 26 includes an X-ray controller 28 that provides power and timing signals to an X-ray source 14, a collimator controller 29 that controls a length and a width of an aperture of the pre-patient collimator 13 (and, thus, the size and shape of the beam of X-rays 16), and a gantry motor controller 30 that controls the rotational speed and position of gantry 12. An image reconstructor 34 receives sampled and digitized X-ray data from DAS 32 and performs high-speed image reconstruction. The reconstructed image is applied as an input to a computer 36, which stores the image in a storage device 38. Computer 36 also receives commands and scanning parameters from an operator via console 40. An associated display 42 allows the operator to observe the reconstructed image and other data from computer 36. The operator supplied commands and parameters are used by computer 36 to provide control signals and information to DAS 32, X-ray controller 28, collimator controller 29, and gantry motor controller 30. In addition, computer 36 operates a table motor controller 44, which controls a motorized table 46 to position subject 22 and gantry 12. Particularly, table 46 moves portions of subject 22 through a gantry opening or bore 48.

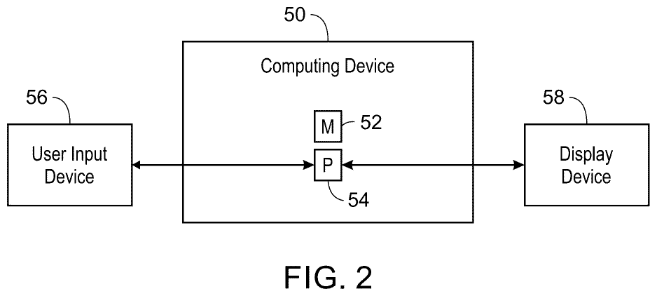

FIG. 2 is a schematic diagram of a computing device 50 for performing the disclosed techniques herein. The computing device 50 may be computer 36 of the CT imaging system 10 in FIG. 1 (or another medical imaging system) or a remote computing device. In certain embodiments, the computing device 50 may be a remote cloud-based processing system.

The computing device 50 includes a memory 52 and a processing system 54. In some embodiments, the processing system 54 may include one or more general purpose processors, one or more application specific integrated circuits, one or more field programmable gate arrays, or the like. Additionally, the memory 52 may be any tangible, non-transitory, computer readable medium that is capable of storing instructions executable by the processing system 54 and/or data that may be processed by the processing system 54. In other words, the memory 52 may include volatile memory, such as random-access memory, or non-volatile memory, such as hard disk drives, read only memory, optical disks, flash memory, and the like. The memory 52 may store imaging data, patient-related data, AEC module having one or more AEC algorithms (e.g., for adjusting exposure setting of the imaging system to obtain a high-quality image), and other data.

The computing device 50 is communicatively coupled with a user input device 56 and a display device 58. The user input device 56 may include one or more of a touchscreen, a keyboard, a mouse, a trackpad, a motion sensing camera, or other device configured to enable a user to interact with the computing device 50. The display device 58 may include one or more display devices utilizing virtually any type of technology. In some embodiments, the display device 58 may include a computer monitor, and may display imaging data and a graphical user interface (e.g., that enables the input a threshold value for much deviation from a target value of a quality or target metric may be accepted by a prescription). The display device 58 may be combined with the processing system 54, the non-transitory memory 52, and/or the user input device 56 in a shared enclosure, or may be peripheral display devices and may comprise a monitor, touchscreen, projector, or other display device known in the art, which may enable a user to view data and/or interact with various data stored in the non-transitory memory 52.

As described in greater detail below, the processing system 54 is configured to receive a prescription (e.g., parameters for conducting a specific scan) for a diagnostic X-ray scan of a subject with a medical imaging scanner, wherein diagnostic X-ray scan has a prescribed value for a target metric. The processing system 54 is configured to determine a predicted value for the target metric for an altered prescription for the diagnostic X-ray scan based on alteration of the prescription by an automatic control exposure algorithm. The processing system 54 is configured to receive an input of a threshold value in a graphical user interface, wherein the threshold value is an acceptable percent deviation between the predicted value and the prescribed value for the target metric. The processing system 54 is configured to determine whether the altered prescription for the diagnostic X-ray scan is sub-optimal based on a comparison of the threshold value to a percent deviation between the predicted value and the prescribed value for the target metric.

In certain embodiments, the processing system 54 is configured to provide a user-perceptible indication on the graphical user interface that the percent deviation exceeds the threshold value for the target metric when the percent deviation exceeds the threshold value for the target metric. In certain embodiments, the user-perceptible indication includes a recommendation to change one or more parameters of the altered prescription to optimize the altered prescription. In certain embodiments, the user-perceptible indication includes an option to accept the altered prescription even though the altered prescription for the diagnostic X-ray scan is sub-optimal, and the processing system 54 is configured to receive user input via the graphical user interface to accept the option. In certain embodiments, the processing system 54 is configured to automatically accept the altered prescription when the percent deviation is equal to or less than the threshold value for the target metric. In certain embodiments, the processing system 54 is configured to provide a user-perceptible prompt on the graphical user interface to accept the altered prescription when the percent deviation is equal to or less than the threshold value for the target metric and receiving user input via the user-perceptible prompt on the graphical user interface to accept the altered prescription.

In certain embodiments, receiving the input of the threshold value in the graphical user interface includes receiving user input of the threshold value. In certain embodiments, the processing system 54 is configured to alter the threshold value inputted via user input to a system threshold value when the threshold value inputted via the user input exceeds the system threshold value, wherein the system threshold value is a value that results in an accepted tolerance for the target metric across all protocols, all series, and all groups for scans with the medical imaging scanner.

In certain embodiments, receiving the input of the threshold value in the graphical user interface includes automatically obtaining a preset threshold value. In certain embodiments, the preset threshold value is a site level threshold value for a site including one or more medical imaging scanners including the medical imaging scanner, a system level threshold value for the medical imaging scanner, a protocol level threshold value specific to a particular medical imaging protocol with the medical imaging scanner, a series level threshold value specific to scanning one or more groups with the particular medical imaging protocol with the medical imaging scanner, or a group level threshold value specific to scanning an individual with the particular medical imaging protocol with the medical imaging scanner. In certain embodiments, the graphical user interface is configurable by a user to present the graphical user interface in a first manner when the percent deviation exceeds the threshold value for the target metric and to present the graphical user interface in a second manner different from the first manner when the percent deviation is equal to or less than the threshold value for the target metric. In certain embodiments, the target metric includes a noise index. In certain embodiments, the target metric includes a ratio between an average tube current of the prescription and an average tube current determined to be optimal by the automatic control exposure algorithm.

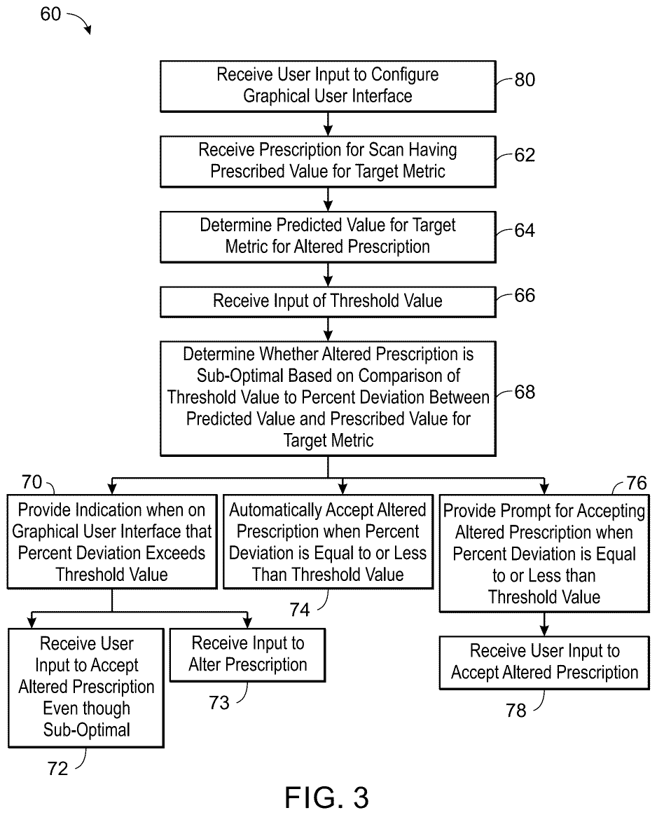

FIG. 3 is a flow chart of a method 60 for thresholding to optimize an X-ray technique. Some or all of the steps of the method 60 may be performed by the computing device 50 in FIG. 2.

The method 60 includes receiving a prescription for a diagnostic X-ray scan (e.g., CT scan) of a subject with a medical imaging scanner (e.g., of a CT imaging system), wherein diagnostic X-ray scan has a prescribed value for a target metric (block 62). In certain embodiments, the target metric may be an image quality metric. In certain embodiments, the target metric may be a noise index. In certain embodiments, the target metric may be target dose. In certain embodiments, the target metric may be target mA or mAs. In certain embodiments, the target metric may be a ratio between an average tube current of the prescription and an average tube current determined to be optimal by the automatic control exposure algorithm. The method 60 also includes determining a predicted value for the target metric for an altered prescription for the diagnostic X-ray scan based on alteration of the prescription by an automatic control exposure algorithm (block 64).

The method 60 further includes receiving an input of a threshold value in a graphical user interface, wherein the threshold value is an acceptable percent deviation between the predicted value and the prescribed value for the target metric (block 66). In certain embodiments, inputs may be received for multiple respective threshold values in the graphical user interface for multiple respective target metrics. In certain embodiments, the threshold value is inputted in a graphical user interface by a user. In certain embodiments, a preset threshold value is automatically obtained. In certain embodiments, the preset threshold value may be a site level threshold value for a site (e.g., hospital or imaging facility or other healthcare facility including one or more medical imaging scanners including the medical imaging scanner). In certain embodiments, the preset threshold value may be a system level threshold value for the medical imaging scanner (e.g., taking into account system constraints). In certain embodiments, the preset threshold value may be a protocol level threshold value specific to a particular medical imaging protocol with the medical imaging scanner. In certain embodiments, the preset threshold value may be a series level threshold value specific to scanning one or more groups with the particular medical imaging protocol with the medical imaging scanner. In certain embodiments, the preset threshold value may be a group level threshold value specific to scanning an individual with the particular medical imaging protocol with the medical imaging scanner. In certain embodiments, the preset threshold value may be task (e.g., clinical use case) specific to the scan being performed (e.g., clinical identifier, age (e.g., adult versus pediatric), anatomical category of protocol, patient size, etc.).

The method 60 further includes determining whether the altered prescription for the diagnostic X-ray scan is sub-optimal (i.e., will not obtain the quality of image) based on a comparison of the threshold value to a percent deviation between the predicted value and the prescribed value for the target metric (block 68). In certain embodiments, the method 60 includes providing a user-perceptible indication on the graphical user interface that the percent deviation exceeds the threshold value for the target metric when the percent deviation exceeds the threshold value for the target metric (block 70). In certain embodiments, the user-perceptible indication includes a recommendation to change one or more parameters of the altered prescription to optimize the altered prescription. In certain embodiments, the user-perceptible indication includes an option (e.g., in the form of a button to click on) to accept the altered prescription even though the altered prescription for the diagnostic X-ray scan is sub-optimal. In this embodiment, the method 60 includes receiving user input via the graphical user interface to accept the option (block 72). In certain embodiments, the method 60 includes receiving user input via the graphical user interface to alter one or more parameters of the prescription (e.g., altered prescription) when the altered prescription is sub-optimal (block 73). In certain embodiments, the method 60 includes automatically accepting the altered prescription when the percent deviation is equal to or less than the threshold value for the target metric (block 74). In certain embodiments, the method 60 includes providing a user-perceptible prompt on the graphical user interface to accept the altered prescription when the percent deviation is equal to or less than the threshold value for the target metric (block 76) and receiving user input via the user-perceptible prompt on the graphical user interface to accept the altered prescription (block 78). The method 60 may include performing one or more of the steps described in conjunction with blocks 70, 74, and 76.

In certain embodiments, the user interface described herein is configurable by a user to present the graphical user interface in a first manner when the percent deviation exceeds the threshold value for the target metric and to present the graphical user interface in a second manner different from the first manner when the percent deviation is equal to or less than the threshold value for the target metric. Thus, in certain embodiments, the method 60 includes receiving user input to configure the graphical user interface when a percent deviation exceeds the threshold value for the target metric and/or to present the graphical user interface in a second manner different from the first manner when the percent deviation is equal to or less than the threshold value for the target metric (block 80).

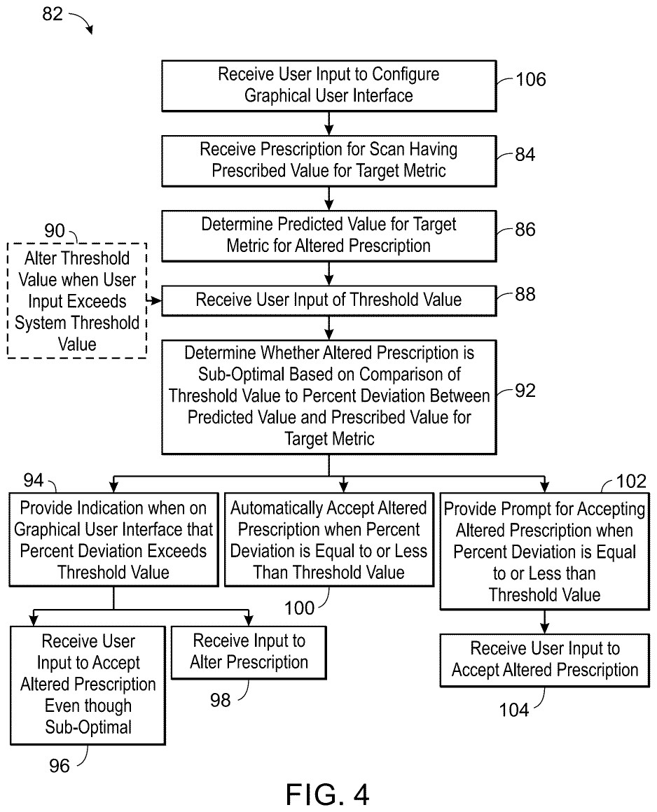

FIG. 4 is a flow chart of a method 82 for thresholding to optimize an X-ray technique (e.g., using user input for threshold value). Some or all of the steps of the method 82 may be performed by the computing device 50 in FIG. 2.

The method 82 includes receiving a prescription for a diagnostic X-ray scan (e.g., CT scan) of a subject with a medical imaging scanner (e.g., of a CT imaging system), wherein diagnostic X-ray scan has a prescribed value for a target metric (block 84). In certain embodiments, the target metric may be an image quality metric. In certain embodiments, the target metric may be a noise index. In certain embodiments, the target metric may be target dose. In certain embodiments, the target metric may be target mA or mAs. In certain embodiments, the target metric may be a ratio between an average tube current of the prescription and an average tube current determined to be optimal by the automatic control exposure algorithm. The method 82 also includes determining a predicted value for the target metric for an altered prescription for the diagnostic X-ray scan based on alteration of the prescription by an automatic control exposure algorithm (block 86).

The method 82 includes receiving a user input of a threshold value in a graphical user interface (in a field on the graphical user interface), wherein the threshold value is an acceptable percent deviation between the predicted value and the prescribed value for the target metric (block 88). In certain embodiments, the user input may be directly entered in the field. In certain embodiments, the threshold value may be selected from a pull-down menu. In certain embodiments, inputs may be received for multiple respective threshold values in the graphical user interface for multiple respective target metrics. In certain embodiments, the method 82 includes altering the threshold value inputted via user input to a system threshold value when the threshold value inputted via the user input exceeds a system threshold value (block 90). The system threshold value may be a value that results in an accepted tolerance for the target metric across all protocols, all series, and all groups for scans with the medical imaging scanner.

The method 82 further includes determining whether the altered prescription for the diagnostic X-ray scan is sub-optimal (i.e., will not obtain the quality of image) based on a comparison of the threshold value to a percent deviation between the predicted value and the prescribed value for the target metric (block 92). In certain embodiments, the method 82 includes providing a user-perceptible indication on the graphical user interface that the percent deviation exceeds the threshold value for the target metric when the percent deviation exceeds the threshold value for the target metric (block 94). In certain embodiments, the user-perceptible indication includes a recommendation to change one or more parameters of the altered prescription to optimize the altered prescription. In certain embodiments, the user-perceptible indication includes an option (e.g., in the form of a button to click on) to accept the altered prescription even though the altered prescription for the diagnostic X-ray scan is sub-optimal. In this embodiment, the method 82 includes receiving user input via the graphical user interface to accept the option (block 96). In certain embodiments, the method 82 includes receiving user input via the graphical user interface to alter one or more parameters of the prescription (e.g., altered prescription) when the altered prescription is sub-optimal (block 98). In certain embodiments, the method 82 includes automatically accepting the altered prescription when the percent deviation is equal to or less than the threshold value for the target metric (block 100). In certain embodiments, the method 82 includes providing a user-perceptible prompt on the graphical user interface to accept the altered prescription when the percent deviation is equal to or less than the threshold value for the target metric (block 102) and receiving user input via the user-perceptible prompt on the graphical user interface to accept the altered prescription (block 104). The method 82 may include performing one or more of the steps described in conjunction with blocks 94, 100, and 102.

In certain embodiments, the user interface described herein is configurable by a user to present the graphical user interface in a first manner when the percent deviation exceeds the threshold value for the target metric and to present the graphical user interface in a second manner different from the first manner when the percent deviation is equal to or less than the threshold value for the target metric. Thus, in certain embodiments, the method 82 includes receiving user input to configure the graphical user interface when a percent deviation exceeds the threshold value for the target metric and/or to present the graphical user interface in a second manner different from the first manner when the percent deviation is equal to or less than the threshold value for the target metric (block 106).

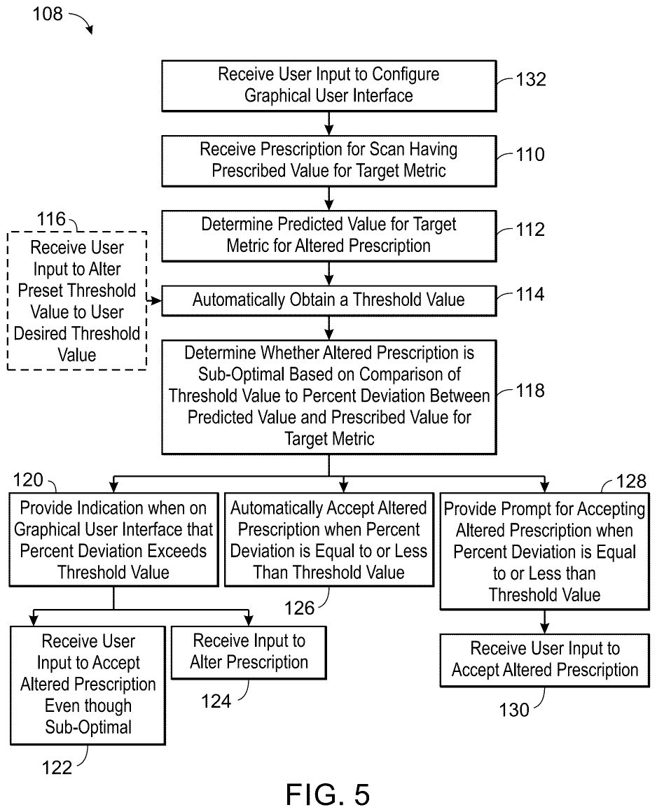

FIG. 5 is a flow chart of a method 108 for thresholding to optimize an X-ray technique (e.g., using preset threshold value). Some or all of the steps of the method 82 may be performed by the computing device 50 in FIG. 2.

The method 108 includes receiving a prescription for a diagnostic X-ray scan (e.g., CT scan) of a subject with a medical imaging scanner (e.g., of a CT imaging system), wherein diagnostic X-ray scan has a prescribed value for a target metric (block 110). In certain embodiments, the target metric may be an image quality metric. In certain embodiments, the target metric may be a noise index. In certain embodiments, the target metric may be target dose. In certain embodiments, the target metric may be target mA or mAs. In certain embodiments, the target metric may be a ratio between an average tube current of the prescription and an average tube current determined to be optimal by the automatic control exposure algorithm. The method 108 also includes determining a predicted value for the target metric for an altered prescription for the diagnostic X-ray scan based on alteration of the prescription by an automatic control exposure algorithm (block 112).

The method 108 includes automatically obtaining a preset threshold value for a threshold value, wherein the threshold value is an acceptable percent deviation between the predicted value and the prescribed value for the target metric (block 114). A field on a graphical user interface may be automatically populated with (or inputted into) the obtained preset threshold value. In certain embodiments, inputs may be received for multiple respective threshold values in the graphical user interface for multiple respective target metrics. In certain embodiments, the preset threshold value may be based on current system learning. For example, the system, similar to intelligent protocoling, may utilize past user behavior to set a threshold and modify that. This learned behavior may be by total usage, by protocol, and/or by clinical context.

In certain embodiments, the preset threshold value may be a site level threshold value for a site (e.g., hospital or imaging facility or other healthcare facility including one or more medical imaging scanners including the medical imaging scanner). In certain embodiments, the preset threshold value may be a system level threshold value for the medical imaging scanner (e.g., taking into account system constraints). In certain embodiments, the preset threshold value may be a protocol level threshold value specific to a particular medical imaging protocol with the medical imaging scanner. In certain embodiments, the preset threshold value may be a series level threshold value specific to scanning one or more groups with the particular medical imaging protocol with the medical imaging scanner. In certain embodiments, the preset threshold value may be a group level threshold value specific to scanning an individual with the particular medical imaging protocol with the medical imaging scanner. In certain embodiments, the preset threshold value may be task (e.g., clinical use case) specific to the scan being performed (e.g., clinical identifier, age (e.g., adult versus pediatric), anatomical category of protocol, patient size, etc.). In certain embodiments, the method 108 includes receiving a user input via the graphical user interface to alter the preset threshold value to a user desired threshold value (as long as the user desired threshold is within the system threshold value) (block 116). The system threshold value may be a value that results in an accepted tolerance for the target metric across all protocols, all series, and all groups for scans with the medical imaging scanner.

The method 108 further includes determining whether the altered prescription for the diagnostic X-ray scan is sub-optimal (i.e., will not obtain the quality of image) based on a comparison of the threshold value to a percent deviation between the predicted value and the prescribed value for the target metric (block 118). In certain embodiments, the method 108 includes providing a user-perceptible indication on the graphical user interface that the percent deviation exceeds the threshold value for the target metric when the percent deviation exceeds the threshold value for the target metric (block 120). In certain embodiments, the user-perceptible indication includes a recommendation to change one or more parameters of the altered prescription to optimize the altered prescription. In certain embodiments, the user-perceptible indication includes an option (e.g., in the form of a button to click on) to accept the altered prescription even though the altered prescription for the diagnostic X-ray scan is sub-optimal. In this embodiment, the method 108 includes receiving user input via the graphical user interface to accept the option (block 122). In certain embodiments, the method 108 includes receiving user input via the graphical user interface to alter one or more parameters of the prescription (e.g., altered prescription) when the altered prescription is sub-optimal (block 124). In certain embodiments, the method 108 includes automatically accepting the altered prescription when the percent deviation is equal to or less than the threshold value for the target metric (block 126). In certain embodiments, the method 108 includes providing a user-perceptible prompt on the graphical user interface to accept the altered prescription when the percent deviation is equal to or less than the threshold value for the target metric (block 128) and receiving user input via the user-perceptible prompt on the graphical user interface to accept the altered prescription (block 130). The method 108 may include performing one or more of the steps described in conjunction with blocks 120, 126, and 128.

In certain embodiments, the user interface described herein is configurable by a user to present the graphical user interface in a first manner when the percent deviation exceeds the threshold value for the target metric and to present the graphical user interface in a second manner different from the first manner when the percent deviation is equal to or less than the threshold value for the target metric. Thus, in certain embodiments, the method 108 includes receiving user input to configure the graphical user interface when a percent deviation exceeds the threshold value for the target metric and/or to present the graphical user interface in a second manner different from the first manner when the percent deviation is equal to or less than the threshold value for the target metric (block 132).

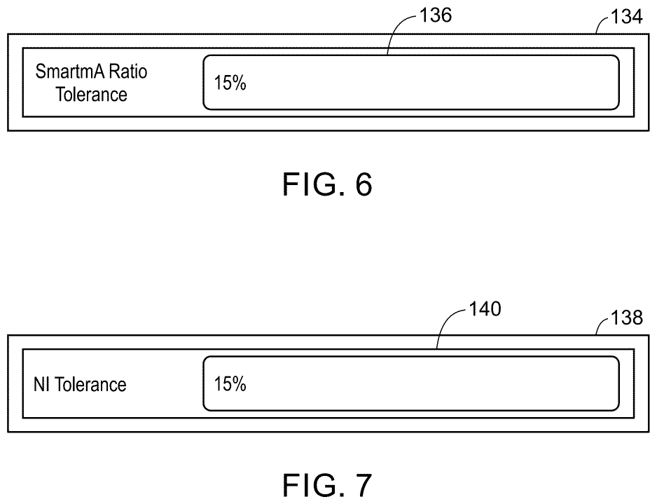

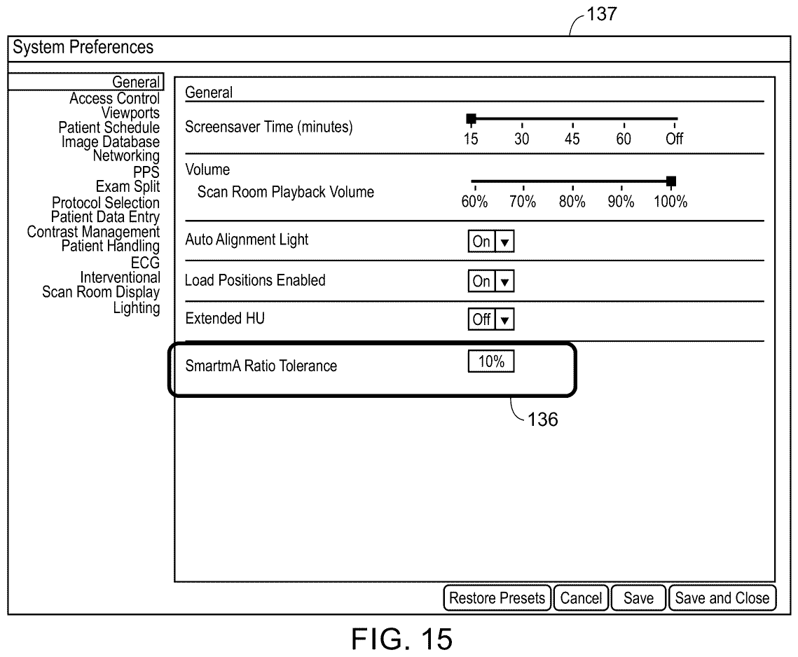

FIG. 6 is a portion of a graphical user interface 134 illustrating a field 136 for a threshold value. As depicted, the target metric relates to tube current. In particular, the field 136 for threshold value represents a ratio between an average tube current of the prescription and an average tube current determined to be optimal by the automatic control exposure algorithm. It is referred to as SmartmA Ratio but may have a different name. As depicted, the threshold value is an acceptable percent deviation between the predicted value and the prescribed value for the target metric (i.e., SmartmA Ratio). FIG. 15 depicts an example of a graphical user interface 137 having the field 136 for the threshold value in FIG. 6.

FIG. 7 is a portion of a graphical user interface 138 illustrating a field 140 for a threshold value. As depicted, the target metric is noise index. In particular, the field 136 for threshold value represents noise index tolerance. As depicted, the threshold value is an acceptable percent deviation between the predicted value and the prescribed value for the target metric (i.e., noise index).

In both FIGS. 6 and 7, in general, the threshold values represent tolerance levels that the site accepts to vary from the target value. The system may not provide any indication that a setting is out of range if it is within the tolerance level from the prescribed target. As noted above, in certain embodiments, a user may input the threshold value. In certain embodiments, the threshold value may be automatically obtained (e.g., obtained from a system preference) and populated into the field for the threshold value. In certain embodiments, the threshold value may be set and/or modified based on learned user behavior (e.g., total usage, by protocol, by clinical context, etc.).

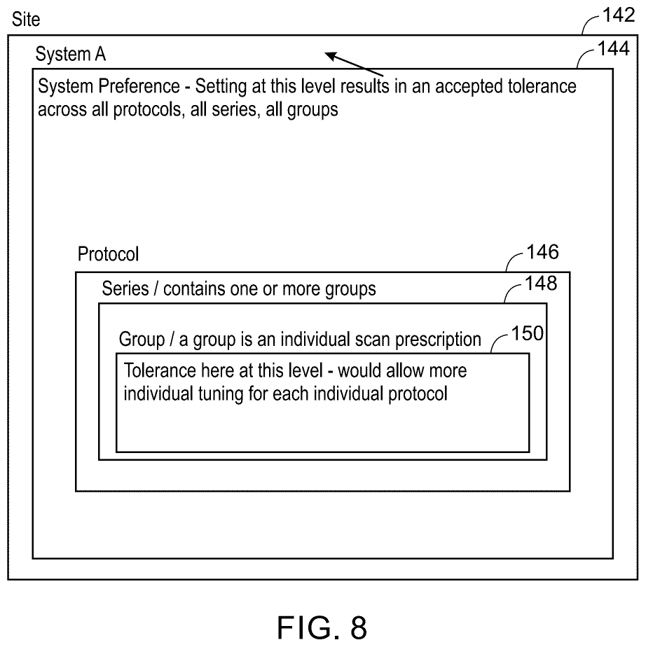

FIG. 8 is a schematic diagram illustrating different sources for setting a threshold value. In certain embodiments, a preset threshold value that is automatically obtained. As depicted, in certain embodiments, the preset threshold value may be a site level threshold value for a site (e.g., hospital or imaging facility or other healthcare facility including one or more medical imaging scanners including the medical imaging scanner) indicated by reference numeral 142. In certain embodiments, the preset threshold value may be a system level threshold value for the medical imaging scanner (e.g., taking into account system constraints) indicated by reference numeral 144. A system preference for the threshold value (at a site (fleet) level or system level) is a value that results in an accepted tolerance for the target metric across all protocols, all series, and all groups for scans with the medical imaging scanner. In certain embodiments, the preset threshold value may be a protocol level threshold value specific to a particular medical imaging protocol with the medical imaging scanner indicated by reference numeral 146. In certain embodiments, the preset threshold value may be a series level threshold value specific to scanning one or more groups with the particular medical imaging protocol with the medical imaging scanner as indicated by reference numeral 148. In certain embodiments, the preset threshold value may be a group level threshold value specific to scanning an individual with the particular medical imaging protocol with the medical imaging scanner as indicate by reference numeral 150. In certain embodiments, the preset threshold value may be task (e.g., clinical use case) specific to the scan being performed (e.g., clinical identifier, age (e.g., adult versus pediatric), anatomical category of protocol, patient size, etc.). For example, with regard to age, dose settings may have a lower tolerance for pediatric patients or, in some cases, a tolerance may not be used at all.

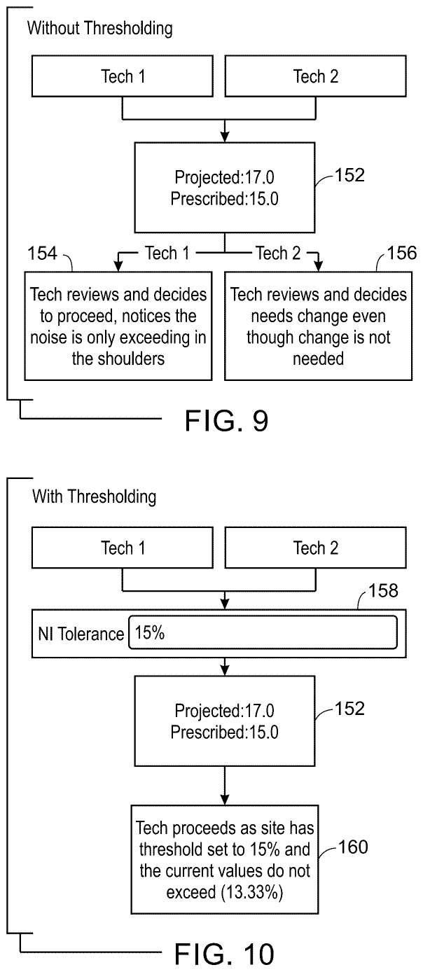

When an alert or notification state is always indicated to the user (e.g., on a graphical user interface), it can put the system at risk due to fatigue of users. A user error may occur such as a prescription (e.g., altered prescription) may not be changed when a target metric (e.g., noise index or SmartmA Ratio) falls outside a tolerance. Conversely, a change may be made when it is not necessary. This scenario is illustrated in FIG. 9. The same projected and prescribed noise index values (indicated by reference numeral 152) are presented to two different technicians. The first technician (as indicated by reference numeral 154) reviews the noise index values and decides to proceed, while the second technician (as indicated by reference numeral 156) reviews the same noise index values and decides the prescription needs a change although a change is not needed.

Providing consistency across a population of technicians can be challenging. Not only is there a gap in experience but often in the modern healthcare world there are travelers and staffing shortages. Implementing a threshold (e.g., by site) and/or the ability to create an action based on this threshold can provide some consistency. FIG. 10 presents the same scenario but with thresholding implemented. The same projected and prescribed noise index values (indicated by reference numeral 152) are presented to two different technicians. However, a threshold value for noise index tolerance is inputted as indicated by reference numeral 158. In this scenario, both technicians proceed with the altered prescription as the site has the threshold set to 15 percent and the current values (with a 13.33 percent difference) do not exceed the threshold value as indicated by reference numeral 160.

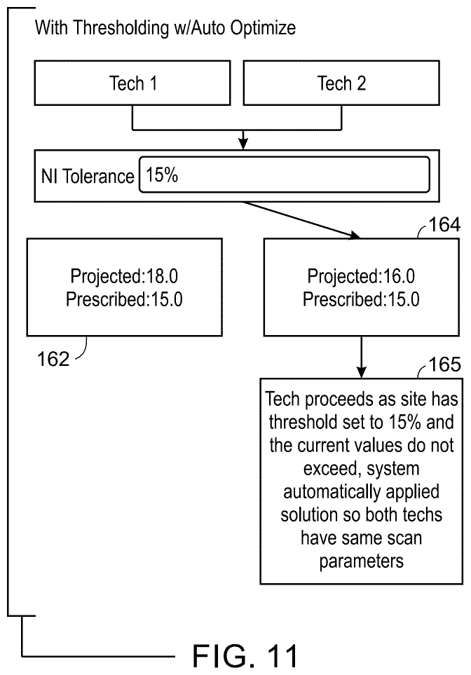

FIG. 11 presents a slightly different scenario from the scenario in FIG. 10. In the scenario in FIG. 11, the two different techs are presented with two different projected noise index values as indicated by reference numerals 162 and 164. However, automatic optimization was applied so that both technicians have the same scan parameters (and same projected noise index as indicated by reference numeral 164). In this scenario, both technicians proceed with the altered prescription as the site has the threshold set to 15 percent and the current values do not exceed the threshold value as indicated by reference numeral 165.

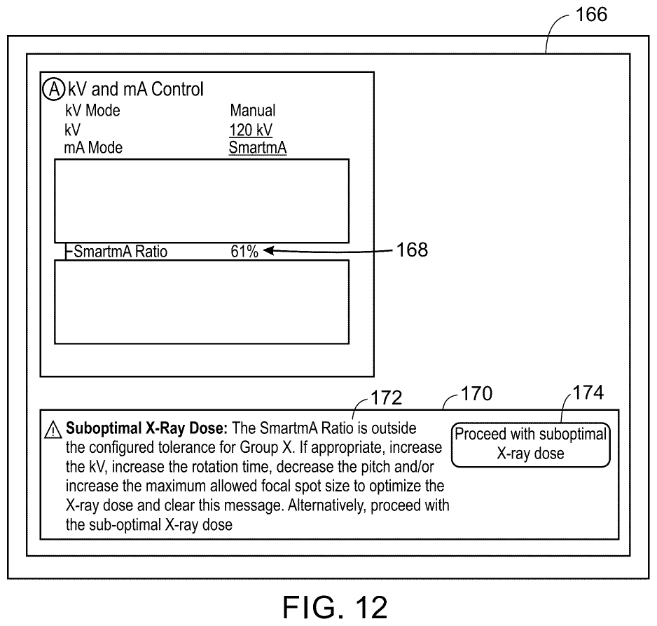

FIG. 12 depicts an example graphical user interface 166 utilizing thresholding and having a recommendation provided. As depicted, the graphical user interface 166 depicts the quality metric (i.e., SmartmA Ratio) as percentage as indicated by reference numeral 168. A user-perceptible notification 170 is presented on the graphical user interface since the quality metric exceeds a configured tolerance or threshold value for that quality metric. The user-perceptible notification 170 includes a recommendation 172 to alter parameters of the prescription. The user-perceptible notification 170 also provides a button 174 that the user may select to proceed with the suboptimal X-ray dose. The user-perceptible notification 170 is an example of a stop implementation since the quality metric was outside the threshold.

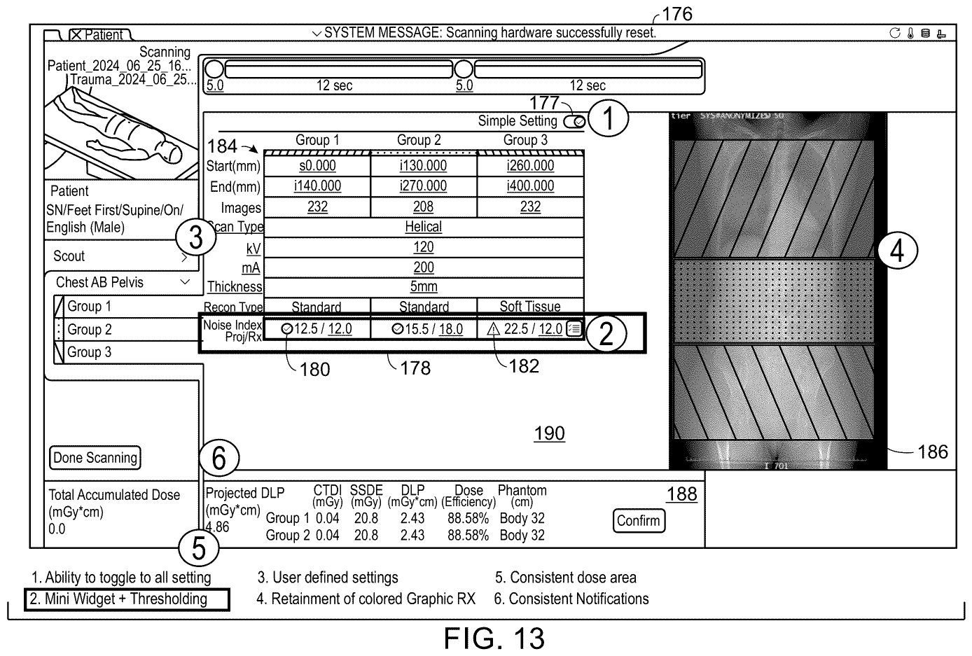

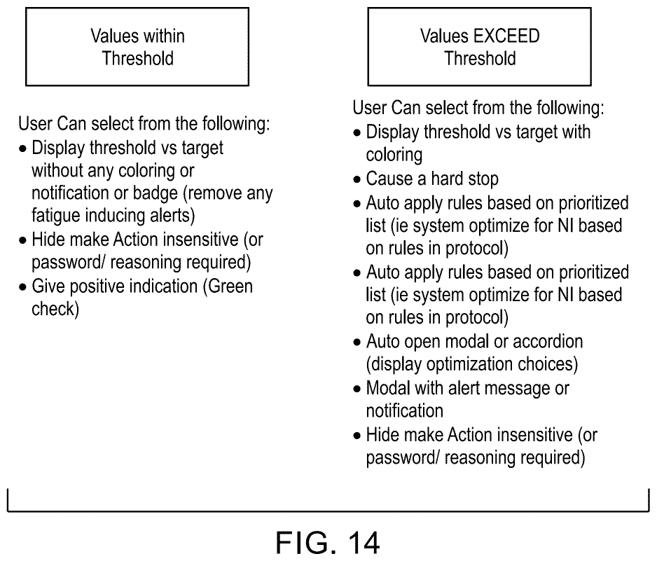

FIG. 13 depicts an example graphical user interface 176 utilizing threshold and its features. The appearance and configuration of the graphical user interface 176 may vary from that depicted in FIG. 13. Features of the graphical user interface 176 include the ability to toggle to all settings as indicated by reference numeral 177. Features of the graphical user interface 176 also include providing a mini widget and thresholding as highlighted by box 178. As depicted in FIG. 13, when the percent deviation between the predicted value and the prescribed value for the target metric (i.e., noise index) is less than or equal to the threshold value, a check mark 180 is provided adjacent the values. However, when the percent deviation between the predicted value and the prescribed value for the target metric (i.e., noise index) exceeds the threshold value, a warning indicator 182 is provided adjacent the values. Features of the graphical user interface 176 also include providing user defined settings 184. These settings 184 may be altered by the user when an altered prescription when a target metric is not within a tolerance level. The minimum settings include scan range, kV, mA, ASIR-V, scan type, thickness images, and time. Features of the graphical user interface 176 also includes retaining colored graphic prescription (RX) as indicated by reference numeral 186. Features of the graphical user interface 176 also include providing a consistent dose area indicated by reference numeral 188. Features of the graphical user interface 176 also include providing an area 190 for consistent notifications. The view for the graphical user interface 176 is determined through a profile editor that provides the user the ability to customize the settings and selected on use case basis per series.

FIG. 14 is a schematic diagram of different levels of configuration for a graphical user interface that utilizes thresholding. In particular, the graphical user interface is configurable by a user to present the graphical user interface in a first manner when the percent deviation exceeds the threshold value for the target metric and to present the graphical user interface in a second manner different from the first manner when the percent deviation is equal to or less than the threshold value for the target metric.

The left side of FIG. 14 depicts different types of configurations that may be selected by the user for when values are within the threshold. For example, a user can select between displaying a threshold versus a target without any coloring or notification or badge, thus, reducing any fatigue inducing alerts. A user may choose to hide or make action insensitive (or requite password or reasoning). A user may choose for a positive indication (e.g., green check) to be provided).

The right side of FIG. 14 depicts different types of configuration that may be selected by the user for when values exceed the threshold. For example, a user can select displaying a threshold versus target with coloring. The user can select for a hard stop to be caused (see example in FIG. 12). The user can select automatic application of rules based on prioritized list (i.e., system optimizes for noise index based on rules in a protocol). The user can select open or accordion (for displaying optimization choices). The user can select providing a modal with alert message or notification. The user may choose to hide or make action insensitive (or requite password or reasoning).

Technical effects of the disclosed embodiments include providing the user control over how much of a deviation from a target value of a quality metric or target metric may be accepted by a prescription (via a threshold value provided on a user interface). Technical effects of the disclosed embodiments include providing a streamlined workflow. Technical effects of the disclosed embodiments include reducing the time to interpret the output of AEC algorithms. Technical effects of the disclosed embodiments include providing a higher consistency in the interpretation of the output of the AEC algorithms among different operators. Technical effects of the disclosed embodiments include providing a higher consistency in an individual operator's interpretation of the output of AEC algorithms.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A method for thresholding to optimize an X-ray technique, comprising:
receiving, via a processing system comprising one or more processors, a prescription for a diagnostic X-ray scan of a subject with a medical imaging scanner, wherein diagnostic X-ray scan has a prescribed value for a target metric;
determining, via the processing system, a predicted value for the target metric for an altered prescription for the diagnostic X-ray scan based on alteration of the prescription by an automatic control exposure algorithm;
receiving, via the processing system, an input of a threshold value in a graphical user interface, wherein the threshold value is an acceptable percent deviation between the predicted value and the prescribed value for the target metric; and
determining, via the processing system, whether the altered prescription for the diagnostic X-ray scan is sub-optimal based on a comparison of the threshold value to a percent deviation between the predicted value and the prescribed value for the target metric.

2. The method of claim 1, further comprising providing, via the processing system, a user-perceptible indication on the graphical user interface that the percent deviation exceeds the threshold value for the target metric when the percent deviation exceeds the threshold value for the target metric.

3. The method of claim 2, wherein the user-perceptible indication comprises a recommendation to change one or more parameters of the altered prescription to optimize the altered prescription.

4. The method of claim 2, wherein the user-perceptible indication comprises an option to accept the altered prescription even though the altered prescription for the diagnostic X-ray scan is sub-optimal, and the computer-implemented method further comprises receiving user input via the graphical user interface to accept the option.

5. The method of claim 2, further comprising automatically accepting, via the processing system, the altered prescription when the percent deviation is equal to or less than the threshold value for the target metric.

6. The method of claim 2, further comprising:
providing, via the processing system, a user-perceptible prompt on the graphical user interface to accept the altered prescription when the percent deviation is equal to or less than the threshold value for the target metric; and
receiving, via the processing system, user input via the user-perceptible prompt on the graphical user interface to accept the altered prescription.

7. The method of claim 1, wherein receiving the input of the threshold value in the graphical user interface comprises receiving, via the processing system, user input of the threshold value.

8. The method of claim 7, further comprising altering, via the processing system, the threshold value inputted via user input to a system threshold value when the threshold value inputted via the user input exceeds a system threshold value, wherein the system threshold value is a value that results in an accepted tolerance for the target metric across all protocols, all series, and all groups for scans with the medical imaging scanner.

9. The method of claim 1, wherein receiving the input of the threshold value in the graphical user interface comprises automatically obtaining, via the processing system, a preset threshold value.

10. The method of claim 9, wherein the preset threshold value is a site level threshold value for a site comprising one or more medical imaging scanners including the medical imaging scanner, a system level threshold value for the medical imaging scanner, a protocol level threshold value specific to a particular medical imaging protocol with the medical imaging scanner, a series level threshold value specific to scanning one or more groups with the particular medical imaging protocol with the medical imaging scanner, or a group level threshold value specific to scanning an individual with the particular medical imaging protocol with the medical imaging scanner.

11. The method of claim 1, wherein the graphical user interface is configurable by a user to present the graphical user interface in a first manner when the percent deviation exceeds the threshold value for the target metric and to present the graphical user interface in a second manner different from the first manner when the percent deviation is equal to or less than the threshold value for the target metric.

12. The method of claim 1, wherein the target metric comprises a noise index.

13. The method of claim 1, wherein the target metric comprises a ratio between an average tube current of the prescription and an average tube current determined to be optimal by the automatic control exposure algorithm.

14. A system for thresholding to optimize an X-ray technique, comprising:
a medical imaging system, the medical imaging system including:
a memory encoding processor-executable routines; and
a processing system comprising one or more processors and configured to access the memory and to execute the processor-executable routines, wherein the processor-executable routines, when executed by the processing system, cause the processing system to:
receive a prescription for a diagnostic X-ray scan of a subject with a medical imaging scanner, wherein diagnostic X-ray scan has a prescribed value for a target metric;
determine a predicted value for the target metric for an altered prescription for the diagnostic X-ray scan based on alteration of the prescription by an automatic control exposure algorithm;
receive an input of a threshold value in a graphical user interface, wherein the threshold value is an acceptable percent deviation between the predicted value and the prescribed value for the target metric; and
determine whether the altered prescription for the diagnostic X-ray scan is sub-optimal based on a comparison of the threshold value to a percent deviation between the predicted value and the prescribed value for the target metric.

15. The system of claim 14, wherein the processor-executable routines, when executed by the processing system, further cause the processing system to provide a user-perceptible indication on the graphical user interface that the percent deviation exceeds the threshold value for the target metric when the percent deviation exceeds the threshold value for the target metric.

16. The system of claim 15, wherein the user-perceptible indication comprises a recommendation to change one or more parameters of the altered prescription to optimize the altered prescription.

17. The system of claim 15, automatically accepting the altered prescription when the percent deviation is equal to or less than the threshold value for the target metric.

18. The system of claim 15, wherein the processor-executable routines, when executed by the processing system, further cause the processing system to:
provide a user-perceptible prompt on the graphical user interface to accept the altered prescription when the percent deviation is equal to or less than the threshold value for the target metric; and
receive user input via the user-perceptible prompt on the graphical user interface to accept the altered prescription.

19. The system of claim 14, wherein the graphical user interface is configurable by a user to present the graphical user interface in a first manner when the percent deviation exceeds the threshold value for the target metric and to present the graphical user interface in a second manner different from the first manner when the percent deviation is equal to or less than the threshold value for the target metric.

20. A non-transitory computer-readable medium, the computer-readable medium comprising processor-executable code that when executed by a processing system comprising one or more processors, causes the processing system to:
receive a prescription for a diagnostic X-ray scan of a subject with a medical imaging scanner, wherein diagnostic X-ray scan has a prescribed value for a target metric;
determine a predicted value for the target metric for an altered prescription for the diagnostic X-ray scan based on alteration of the prescription by an automatic control exposure algorithm;
receive an input of a threshold value in a graphical user interface, wherein the threshold value is an acceptable percent deviation between the predicted value and the prescribed value for the target metric; and
determine whether the altered prescription for the diagnostic X-ray scan is sub-optimal based on a comparison of the threshold value to a percent deviation between the predicted value and the prescribed value for the target metric.
